(19) United States
(12) Patent Application Publication
(10) Pub. No.: US 2004/0010172 A1
(43) Pub. Date: Jan. 15, 2004

(54) Process for preparing styrene

(76) Inventors: Andrew David Horton, Amsterdam (NL); Timothy Michael Nisbet, Amsterdam (NL); Marinus Van Zwienen, Amsterdam (NL)

Correspondence Address: Jennifer D. Adamson, Shell Oil Company, Legal - Intellectual Property, P. O. Box 2463, Houston, TX, 77252-2463, US

(21) Appl. No.: 10/601,049

(22) Filed: Jun. 20, 2003

(30) Foreign Application Priority Data
Jun. 21, 2002 (EP) 02254359.9

(51) Int. Cl. C07C 1/207; C07C 5/09; C07C 15/40

(52) U.S. Cl. 585/436; 585/435

(57) ABSTRACT

The invention relates to a process for preparing styrene by catalytically dehydrating 1-phenylethanol in the liquid phase in the presence of at least 0.1 % wt of a chain transfer agent. As an alternative, the process is performed by recycling at least part of the chain transfer-containing product stream to the reactor and combining with the feed for preparing the products.

### FIELD OF THE INVENTION

The invention relates to a process for preparing styrene by catalytically dehydrating 1-phenylethanol in the liquid phase.

### BACKGROUND OF THE INVENTION

Such processes are known in the art, for example from U.S. Pat. No. 3,526,674 which describes the dehydration of α-phenylethanol (1-phenyl ethanol) to form styrene in the liquid phase at temperatures above 200° C. This process also provides considerable amounts of ethyl benzene-like byproducts that are distilled off. This distillation process is expensive because the boiling points of product and byproduct are close. Further, in addition to relatively low-boiling ethyl benzene byproducts, oligomers and polymers are formed that are generally indicated as heavy condensation products or “heavies.” More recently, U.S. Pat. No. 5,639,928 disclosed a process for suppressing such undesirable heavy formation when making styrene monomer by catalytically dehydrating 1-phenyl ethanol. It was found that residue formation is suppressed by performing the reaction in the presence of a nitro group-containing additive.

The synthesis of styrene is important because this product functions as the starting material for valuable commercial products such as plastics and the like. The above disadvantages of these processes contribute considerably to the cost of the styrene starting material. These cost contributions arise not only due to the quantities of byproducts obtained, but also due to the properties thereof. Particularly, viscous high-molecular byproducts contribute substantially to the purification costs, whereas low-viscous low-molecular byproducts are much easier to remove.

It would be useful to provide a new process resulting in byproducts that are less viscous and have lower molecular weight than the byproduct obtained with the processes of the prior art.

The effect of phenol in the dehydration of 1-phenylethanol is unclear. U.S. Pat. No. 3,442,963 describes that phenol is advantageous for dehydration of 1-phenylethanol in the gas phase while U.S. Pat. No. 5,171,868 and U.S. Pat. No. 5,538,599 warn against the presence of phenol in the dehydration of 1-phenylethanol. Dehydration is generally carried out in the gas phase as mentioned and taught in U.S. Pat. No. 3,351,635, referred to by both U.S. Pat. No. 5,171,868 and U.S. Pat. No. 5,538,599.

### SUMMARY OF THE INVENTION

It has now been found that low viscous byproducts having relatively low molecular weight are obtained when a chain transfer agent is present in the liquid phase reaction mixture to be dehydrated. Thus the invention pertains to the process for preparing styrene by catalytically dehydrating 1-phenylethanol in the liquid phase, wherein the process is performed in the presence of at least 0.1% wt of a chain transfer agent. It is stressed that the amount of heavies obtained is not necessarily decreased, but the improvement is particularly found in the decreased viscosity and molecular weight thereof.

### DETAILED DESCRIPTION OF THE INVENTION

The amount of chain transfer agent present in a conventional 1-phenylethanol feed for liquid phase dehydration depends on the exact process conditions applied and catalyst used. Typically, the amount of chain transfer agent will be less than 0.1% wt.

Dehydration conditions of temperature and pressure as well as the selection of the catalyst are generally known in the field. Such procedure generally involves dehydrating 1-phenylethanol in the liquid phase at temperatures ranging from about 150° C. to about 350° C., preferably from about 180° C. to about 280° C., and more preferably from about 200° C. to 260° C. The pressure generally is sub-atmospheric to atmospheric, from about 0.05 bar to about 1 bar, preferably from about 0.2 bar to about 0.6 bar, and more preferably from about 0.3 bar to about 0.5 bar.

Acidic type catalysts are preferably employed such as aliphatic and aromatic sulfonic acids. Examples are oxalic acid, sulfuric acid, and particularly p-toluene sulfonic acid.

An important feature of the present invention is the use of at least 0.1% wt of a chain transfer agent. The usual chain transfer agents can be employed, such as those disclosed in the reference work by K. C. Berger and G. Brandrup, Polymer Handbook (ed. J. Brandrup and E. H. Immergut; 3rd ed. John Wiley & Sons), Transfer Constants to Monomer, Polymer, catalyst, Solvent, and Additive in Free Radical Polymerization, II, pp. 81-151. The precise function of the chain transfer agent in the present invention is not yet fully understood. Without wishing to be bound to any theory, it is thought that the chain transfer agent might act as an inhibitor for styrene polymerization as well. A general description of suitable compounds for use in the present invention would be an aromatic compound containing a hydroxyl group which is either linked directly to the aromatic ring or as part of an alkyl group containing a single carbon atom. Besides the hydroxyl group or alkyl group containing hydroxyl, the aromatic ring can contain further alkyl substituents containing of from 1 to 4 carbon atoms. The alkyl group containing hydroxyl can be a carboxylic acid. Particularly useful chain transfer agents are selected from phenol, methylphenol, ethylphenol, benzylalcohol and benzoic acid. Of course, mixtures of at least two different chain transfer agents can also be used. In practice, the amount of chain transfer agent used is generally between about 0.1% wt and about 10% wt. Preferred amounts of chain transfer agent range between about 0.3% wt and about 5% wt, most preferably between about 0.8% wt and about 1.5% wt.

An interesting embodiment of the invention is to use the crude product of the above reaction as a source of the chain transfer agent. In a commonly used process for preparing 1-phenylethanol, ethyl benzene is first oxidized with air to form ethylbenzene hydroperoxide. This is then reacted with propene to yield propylene oxide and 1-phenylethanol (K. Weissermel and H-J Arpe, Industrial Organic Chemistry, Third edition (1997) VCH Verslaggesellschaft GmbH, pp. 267-270). In this process, small amounts of compounds such as benzylalcohol, phenol, and ethylphenols are formed as a byproduct. Thus, the crude 1-phenylethanol stream as such contains amounts of chain transfer agent as the result of a side-reaction. In a conventional process, process steps are carried out to remove these components. For example, phenol is removed by a caustic wash or distillation (U.S. Pat. No. 5,538,599) and benzaldehyde is removed by distillation. These separation steps may now fully or partially be omitted and (part of) the product stream of the dehydration reaction may be used as the source of chain transfer agent by recycling such product stream to the reactor. The concentration of chain transfer agent in the feed to the dehydration reaction is hereby increased. Thus, only at the start of such process highly viscous byproduct are formed, which is insignificant since these minor amounts are of no influence on the end viscosity after the recycling process has set forth. It is also possible to start with the addition of a chain transfer agent, performing the process and then replacing the addition of the-chain transfer agent by addition of (part of) the product stream. A method of continuously adding both external chain transfer agent and recycled product stream is also possible. The invention thus further pertains to a process for preparing styrene comprising the steps:

a) catalytically dehydrating a feed comprising 1-phenylethanol in the liquid phase in a reactor to obtain a product stream comprising chain transfer agent; and

b) recycling at least part of the chain transfer agent-containing product stream to the reactor to be combined with the feed.

In this manner at least 0.1% wt of the chain transfer agent is added to the feed.

The new process has many advantages over the existing processes since the low viscosity byproducts allow easy handling procedures, wherein the reaction mass is much more easily pumped through the plant. Further, the low viscosity byproducts have better heat exchange properties, requiring less expensive heat exchangers and the like. In the case where the recycle of the dehydration reaction product is used to increase the level of chain transfer agent in the feed, the process has the further advantage that some expensive separation steps are no longer necessary.

The feed for use in the present invention can be prepared in any way known to someone skilled in the art. It has been found to be especially advantageous to use a-feed which has been prepared by contacting propene and ethylbenzene-hydroperoxide in the presence of a heterogeneous catalyst to obtain, propylene;oxide and 1-phenylethanol. Therefore, the present invention also relates to a process for preparing styrene, which process comprises (i) contacting propene and ethylbenzene hydroperbxide in the presence of a heterogeneous catalyst to obtain propylene oxide and 1-phenylethanol, (ii) separating 1-phenylethanol from the reaction mixture obtained in step (i), and (iii) use of 1-phenylethanol obtained in step (ii) in a liquid phase dehydration according to the present invention.

The invention is further illustrated by the following non-limiting examples.

### EXAMPLE 1

A dehydration reaction of 1-phenylethanol was carried out as follows. 64 g of heavy residue from a previous dehydration reaction was placed in a reactor. A mixture of 99% wt 1-phenylethanol (laboratory grade) and 1% wt phenol was continuously fed to the reactor at a rate of 30 g/h. The reactor conditions were 0.6 bar and 225° C. The feed contained 200 mg p-toluene sulfonic acid/kg feed. Reaction products styrene and water were removed as vapor. After 48 h operation, a total of 195 g of heavy residue was present. Average molecular weight of this residue was determined as follows: A solution of the sample was prepared by adding about 3 ml of THF (tetrahydrofuran) (containing 0.1% of elemental sulfur as internal standard) to 10-15 mg heavy-ends at 25° C. and agitating the mixture for 30 min. The samples, together with the reference sample (polystyrene), were then analyzed under the following chromatographic conditions:

| | |
|---|---|
| Column: | PL-Gel, four 30 cm columns (10000, 1000, |
| | 100 and 50 Angstroms), 50 μm |
| Eluent: | tetrahydrofuran |
| Flow rate: | 0.8 ml/min |
| Temperature: | 40° C. |
| Detector: | Refractive index (at 30° C.) |
| Calibration: | Polystyrene standard (266-377400 Da) |

Molecular weight of the heavy residue was as follows: Mn 288, Mw 509, Mz 982.

### COMPARATIVE EXAMPLE 1

A dehydration reaction of 1-phenylethanol was carried out as in Example 1, except that the feed was only 1-phenylethanol (laboratory grade). 70 g of heavy residue were charged to the reactor. After 52 h operation a total of 203 g of heavy residue was present. Molecular weight of the heavy residue was as follows: Mn 404, Mw 6965, Mz 219513.

### EXAMPLE 2

A dehydration reaction of 1-phenylethanol was carried out as in Example 1, except that the feed was a mixture of 99% wt of 1-phenylethanol (laboratory grade) and 1% wt of benzylalcohol. 62 g of heavy residue were charged to the reactor. After 75 h operation a total of 341 g of heavy residue was present. Molecular weight of the heavy residue was as follows: Mn 420, Mw 711, Mz 1165.

We claim:

1. A process for preparing styrene comprising:
catalytically dehydrating 1-phenylethanol in the liquid phase
wherein the process is performed in the presence of at least 0.1% wt of a chain transfer agent.

2. The process of claim 1 in which the chain transfer agent is selected from the group consisting of phenol, methylphenol, ethylphenol, benzylalcohol and benzoic acid.

3. The process of claim 1 in which the process is performed in the presence of an acidic catalyst.

4. The process of claim 3 in which the catalyst is an aliphatic or aromatic sulfonic acid.

5. The process of claim 1 in which the process is performed at. 150° C. to 350° C.

6. A process for preparing styrene comprising:
a) catalytically dehydrating a feed comprising 1-phenylethanol in the liquid phase in a reactor to obtain a product stream comprising chain transfer agent; and,
b) recycling at least part of the chain transfer agent-containing product stream to the reactor to be combined with the feed.

7. A process for preparing styrene comprising:
(i) contacting propene and ethylbenzene hydroperoxide in the presence of a heterogeneous catalyst to obtain propylene oxide and 1-phenylethanol;
(ii) separating 1-phenylethanol from the reaction mixture obtained in step (i); and,
(iii) introducing the 1-phenylethanol obtained in step (ii) into a process for preparing styrene comprising catalytically dehydrating 1-phenylethanol in the liquid phase wherein the process is performed in the presence of at least 0.1% wt of a chain transfer agent.
